(19) United States
(12) Patent Application Publication
(10) Pub. No.: US 2025/0256084 A1
(43) Pub. Date: Aug. 14, 2025

(54) UNIVERSAL HEART PORT DEVICE, SYSTEM, and RELATED METHODS THEREOF

(71) Applicant: University of Virginia Patent Foundation, Charlottesville, VA (US)

(72) Inventors: Leora T. Yarboro, Charlottesville, VA (US); Evan Rotar, Charlottesville, VA (US)

(21) Appl. No.: 19/053,059

(22) Filed: Feb. 13, 2025

Related U.S. Application Data
(60) Provisional application No. 63/553,509, filed on Feb. 14, 2024.

(51) Int. Cl. A61M 39/02; A61M 60/178; A61M 60/863; A61M 60/892

(52) U.S. Cl. CPC A61M 39/0247; A61M 60/178; A61M 60/863; A61M 60/892; A61M 2039/0261

(57) ABSTRACT

A universal heart port, in various embodiments, comprises: a cylindrical heart port body defining a circular top surface, an outer surface, and a cylindrical opening that extends through the heart port body; and one or more adapter coupling flanges, each respective adapter coupling flange of the one or more adapter coupling flanges extending radially outward along at least a portion of the top surface of the heart port body and comprising a respective end stop portion that extends perpendicularly downward from a respective end portion of the respective adapter coupling flange. In some embodiments, each of the adapter coupling flanges are substantially evenly spaced about the outer surface of the heart port body. In some embodiments, the heart port further comprises one or more adapters configured for selective coupling to the heart port. The adapters may include a plug, a canula adapter, an LVAD adapter, etc.

### CROSS-REFERENCE TO RELATED APPLICATION

This application claims the benefit of U.S. Provisional Patent Application Ser. No. 63/553,509, filed Feb. 14, 2024, the disclosure of which is hereby incorporated herein by reference in its entirety.

### BACKGROUND

Reliable and safe access mechanisms for multimodal therapy of complex cardiovascular issues have not been established in the current marketplace. Accordingly, there is a need for improved devices and techniques for providing access to the heart for various intracardiac procedures and other types of procedures.

### SUMMARY

In accordance with the purpose(s) of the present disclosure, as embodied and broadly described herein, the disclosure, in one aspect, relates to a heart port device, system and related methods thereof. Various embodiments can include a universal heart port device, system, and related methods that enable safe access to the heart for lifesaving intracardiac procedures or other types of procedures as desired or required. Still other embodiments provide such access to other anatomical regions.

Other systems, methods, features, and advantages of the present disclosure will be or become apparent to one with skill in the art upon examination of the following drawings and detailed description. It is intended that all such additional systems, methods, features, and advantages be included within this description and be within the scope of the present disclosure. In addition, all optional and preferred features and modifications of the described embodiments are usable in all aspects of the disclosure taught herein.

A universal heart port, in particular embodiments, comprises: (1) a substantially cylindrical heart port body defining a substantially circular top surface, an outer surface, and a substantially cylindrical opening that extends through the heart port body; (2) a suture ring support flange that extends radially outward from the outer surface of the heart port body adjacent a base of the heart port body, the suture ring support flange being configured to support a suture ring adjacent the base of the universal heart port; and (3) one or more adapter coupling flanges, each respective adapter coupling flange of the one or more adapter coupling flanges extending radially outward along at least a portion of the top surface of the heart port body and comprising a respective end stop portion that extends perpendicularly downward from a respective end portion of the respective adapter coupling flange. In some embodiments, each adapter coupling flange is substantially parallel to and spaced apart from the suture ring, and each of the one or more adapter coupling flanges are substantially evenly spaced about the outer surface of the heart port body.

In particular aspects, the universal heart port device of claim 1, further comprises a universal heart port adapter comprising: (1) a substantially cylindrical body portion defining a substantially cylindrical cavity adjacent a base of the universal heart port adapter; and (2) one or more heart port engaging portions disposed at least partially within the cavity, wherein each of the one or more heart port engaging portions comprise a respective flange support shelf configured to engage a respective adapter coupling flange of the one or more adapter coupling flanges and cooperate with the respective adapter coupling flange to at least temporarily couple the universal heart port to the universal heart port adapter. In some embodiments, the universal heart port adapter comprises at least one of a plug, a canula adapter, a left ventricular assist device (LVAD) adapter, an extension ring, or a membrane access port.

In particular embodiment, each of the one or more heart port engaging portions further comprise a respective flange support portion that extends from a base of the cavity to a heart port engaging face disposed adjacent an upper portion of the cavity in an interior of the substantially cylindrical body portion; and each respective adapter coupling flange extends perpendicularly from an end of a respective flange support portion adjacent the base of the cavity such that at least a portion of each respective adapter coupling flange, each respective flange support portion, and the heart port engaging face define a respective flange receiving channel configured to receive at least a portion of a respective adapter coupling flange. In some aspects, each of the one or more heart port engaging portions are spaced apart by a distance that corresponds to a length of each respective adapter coupling flange including the respective end stop portion. In some aspects, each respective flange receiving channel has a length that corresponds to a length of each respective adapter coupling flange without including the respective end stop portion.

In some embodiments, the one or more adapter coupling flanges comprise a first adapter coupling flange and a second adapter coupling flange; and the one or more heart port engaging portions comprise a first heart port engaging portion and a second heart port engaging portion. In various aspects, the first adapter coupling flange is substantially structurally identical to the second adapter coupling flange; and the first hear port engaging portion is substantially structurally identical to the second heart port engaging portion.

A universal heart port device, in any embodiment described herein, may comprise: (1) a base portion comprising: (A) a substantially cylindrical heart port body defining a substantially circular top surface, an outer surface, and a substantially cylindrical membrane cutout that extends through the heart port body; (2) a suture ring support flange that extends radially outward from the outer surface of the heart port body adjacent a base of the heart port body; and (3) one or more adapter coupling flanges, each respective adapter coupling flange of the one or more adapter coupling flanges extending radially outward along at least a portion of the top surface of the heart port body and comprising a respective end stop portion that extends perpendicularly downward from a respective end portion of the respective adapter coupling flange; (B) an adapter comprising: (1) a substantially cylindrical body portion defining a substantially cylindrical cavity adjacent a base of the adapter; and (2) one or more base portion engaging portions disposed at least partially within the cavity, wherein each of the one or more base portion engaging portions comprise a respective flange support shelf configured to engage a respective adapter coupling flange of the one or more adapter coupling flanges and cooperate with the respective adapter coupling flange to at least temporarily couple the base portion to the adapter.

In some embodiments, the adapter comprises at least one of a plug, a canula adapter, a left ventricular assist device (LVAD) adapter, an extension ring, or a membrane access port. In various aspects, each of the one or more base portion engaging portions are spaced apart by a distance that corresponds to a length of each respective adapter coupling flange including the respective end stop portion.

In particular embodiments, each of the one or more base portion engaging portions further comprise a respective flange support portion that extends from a base of the cavity to a base portion engaging face disposed adjacent an upper portion of the cavity in an interior of the substantially cylindrical body portion; and each respective adapter coupling flange extends perpendicularly from an end of a respective flange support portion adjacent the base of the cavity such that at least a portion of each respective adapter coupling flange, each respective flange support portion, and the base portion engaging face define a respective flange receiving channel configured to receive at least a portion of a respective adapter coupling flange. In some aspects, each respective flange receiving channel has a length that corresponds to a length of each respective adapter coupling flange without including the respective end stop portion.

In some embodiments, the one or more base portion engaging portions comprise a first base portion engaging portion and a second base portion engaging portion; and the first base portion engaging portion and the second base portion engaging portion are substantially evenly spaced about an interior of the cavity. In various embodiments, the first base portion engaging portion and the second base portion define: (A) a first flange insertion channel between a first end of the first base portion engaging portion and a second end of the second base portion engaging portion; and (B) a second flange insertion channel between a second end of the first base portion engaging portion and a first end of the second base portion engaging portion. In some embodiments, each of the first flange insertion channel and the second flange insertion channel are dimensioned to receive a respective adapter coupling flange.

A method of affixing a universal heart port to a heart, in various embodiments, comprises: (A) providing a universal heart port comprising: (1) a substantially cylindrical heart port body defining a substantially circular top surface, an outer surface, and a substantially cylindrical opening that extends through the heart port body; (2) a suture ring support flange that extends radially outward from the outer surface of the heart port body adjacent a base of the heart port body, the suture ring support flange supporting a suture ring that extends radially outward beyond an outer portion of the suture ring support flange; and (3) one or more adapter coupling flanges, each respective adapter coupling flange of the one or more adapter coupling flanges extending radially outward along at least a portion of the top surface of the heart port body and comprising a respective end stop portion that extends perpendicularly downward from a respective end portion of the respective adapter coupling flange; (B) using the suture ring to suture the universal heart port to the heart; and (C) creating an incision on the heart within at least a portion of the substantially cylindrical opening of the universal heart port.

In some embodiments, the method further comprises providing a universal heart port adapter comprising: (1) a substantially cylindrical body portion defining a substantially cylindrical cavity adjacent a base of the universal heart port adapter; and (2) one or more heart port engaging portions disposed at least partially within the cavity, wherein each of the one or more heart port engaging portions comprise a respective flange support shelf configured to engage a respective adapter coupling flange of the one or more adapter coupling flanges and cooperate with the respective adapter coupling flange to at least temporarily couple the universal heart port to the universal heart port adapter. In such aspects, the method may include at least temporarily coupling the universal heart port adapter to the universal heart port.

In particular embodiments, at least temporarily coupling the universal heart port adapter to the universal heart port comprises: (1) inserting the universal heart port adapter into the universal heart port such that each of the one or more heart port engaging portions pass between adjacent adapter coupling flanges of the one or more adapter coupling flanges; and (2) rotating the universal heart port adapter until each respective end stop portion engages at least a portion of a respective end portion of each respective flange support shelf. In some embodiments, rotating the universal heart port adapter until each respective end stop portion engages at least a portion of the respective end portion of each respective flange support shelf comprises rotating the universal heart port adapter such that each respective adapter coupling flange engages a respective flange support shelf.

In various embodiments: (1) each of the one or more heart port engaging portions further comprise a respective flange support portion that extends from a base of the cavity to a heart port engaging face disposed adjacent an upper portion of the cavity in an interior of the substantially cylindrical body portion; (2) each respective adapter coupling flange extends perpendicularly from an end of a respective flange support portion adjacent the base of the cavity such that at least a portion of each respective adapter coupling flange, each respective flange support portion, and the heart port engaging face define a respective flange receiving channel configured to receive at least a portion of a respective adapter coupling flange; and (3) rotating the universal heart port adapter until each respective end stop portion engages at least a portion of the respective end portion of each respective flange support shelf comprises rotating the universal heart port adapter such that each respective adapter coupling flange is at least partially disposed within a respective flange receiving channel.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

Drawings are presented in the attachment files accompanying this specification:

FIG. 1 is a perspective view of a universal heart port in accordance with various other embodiments of the present disclosure.

FIG. 2 is a side view of the universal heart port shown in FIG. 1.

FIG. 3 is a top view of the universal heart port shown in FIG. 1.

FIG. 4 is a view of a universal heart port adapter in accordance with various embodiments of the present disclosure.

FIG. 5 is a rear perspective view of the universal heart port adapter shown in FIG. 4.

FIG. 6 is a detail view of a flange engaging shelf of a universal heart port adapter in accordance with various embodiments of the present disclosure, for example, such as the universal heart port adapter of FIG. 4.

FIG. 7 is a rear view of the universal heart port adapter shown in FIG. 4.

FIGS. 8-9 are views of a universal heart port adapter in accordance with another embodiment of the present disclosure.

FIGS. 10-11 are views of a universal heart port adapter in accordance with yet another embodiment of the present disclosure.

FIG. 12 is a rear view of the universal heart port adapter shown in FIGS. 10-11.

FIG. 13 is a view of the universal heart port of FIG. 1 sutured to a heart in accordance with various aspect of the present disclosure.

FIG. 14 is a detail view of a process for at least temporarily coupling a universal heart port adapter to a universal heart port.

FIGS. 15A-C depict a process for at least temporarily coupling a universal heart port adapter to a universal heart port.

Additional advantages of the invention will be set forth in part in the description which follows, and in part will be obvious from the description, or can be learned by practice of the invention. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the disclosure. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method and/or process can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a spacer,” “a guide nucleic acid,” or “an miRNA,” including, but not limited to, mixtures or combinations of two or more such spacers, guide nucleic acids, or miRNAs, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Overview

As noted above, there is a need for improved devices and techniques for providing access to the heart for various intracardiac procedures and other types of procedures. In various embodiments, a universal heart port device comprises a universal heart port configured for securing to a heart of a patient. The universal heart port device may then include one or more universal heart port adapters configured to at least temporarily couple to the universal heart port. As may be understood from the examples described herein, various embodiments of universal heart port adapters may provide different functionality. For example, in particular embodiments, the universal hear port adapter may include a plug, a canula adapter, a left ventricular assist device (LVAD) adapter, an extension ring, a membrane access port, or other suitable components.

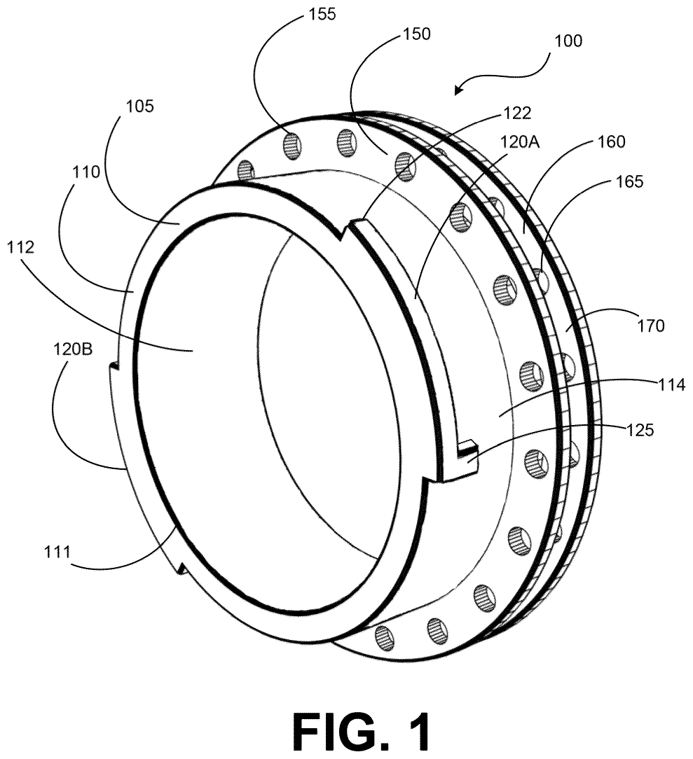

FIG. 1 depicts a universal heart port 100 in accordance with various embodiments of the present disclosure. As may be understood from this figure, the universal heart port 100 comprises a substantially cylindrical body portion 105 that extends from a top surface 110 to a base of the universal heart port 100. The body portion 105 defines a substantially circular membrane access cutout 111 that extends centrally through a length of the body portion 105 and is configured to provide access to a membrane (e.g., surface and/or interior of a heart or other organ to which the universal heart port 100 is attached). The membrane access cutout 111 further defines an interior surface 112.

The universal heart port 100 comprises a first suture ring support flange 150 that extends radially outward around a circumference of an outer surface 114 of the body portion 105. In various embodiments, the first suture ring support flange 150 defines a plurality of anchor cutouts 155 about a circumference of the first suture ring support flange 150. In the embodiment shown in this figure, the plurality of anchor cutouts 155 are substantially evenly spaced about the first suture ring support flange 150.

Similarly, the universal heart port 100 comprises a second suture ring support flange 160 disposed adjacent a base of the universal heart port 100. In various embodiments, the second suture ring support flange 160 is substantially structurally similar to the first suture ring support flange 150. As such, the second suture ring support flange 160 extends radially outward around a circumference of an outer surface 114 of the body portion 105. In various embodiments, the second suture ring support flange 160 defines a plurality of anchor cutouts 165 about a circumference of the second suture ring support flange 160. In the embodiment shown in this figure, the plurality of anchor cutouts 165 are substantially evenly spaced about the second suture ring support flange 160.

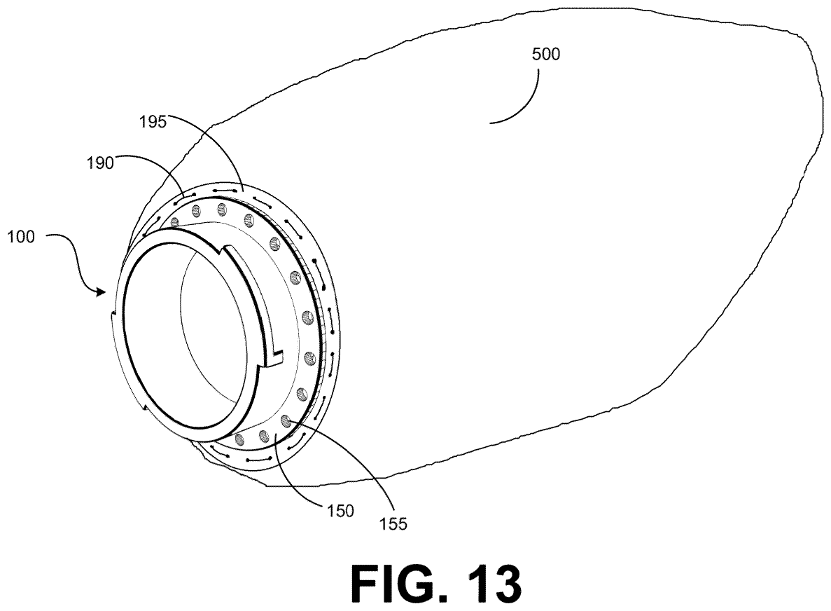

In some embodiments, the first suture ring support flange 150 and the second suture ring support flange 160 are spaced apart about the body portion 105 and define a suture ring receiving channel 170 therebetween. In some aspects, a suture ring 195 (FIG. 13) is disposed at least partially within the suture ring receiving channel 170. In various embodiments, the suture ring 195 is substantially circular and ring-shaped. In various embodiments, a first portion of the suture ring 195 is disposed within the suture ring receiving channel 170 (i.e., between the first suture ring support flange 150 and the second suture ring support flange 160) and a second portion of the suture ring 195 extends radially beyond an outer edge of the first suture ring support flange 150 and the second suture ring support flange 160. In this way, the suture ring 195, in particular embodiments, is adapted for attachment to the heart or other organ/tissue.

In some embodiments, the suture ring 195 serves to attach the universal heart port 100 the heart, and may comprise any suitable material such as polyester velour, felt, expanded polytetrafluoroethylene (EPTFE) or various other suitable fabric. In some aspects, the suture ring 195 is adapted for attachment to the heart with sutures or staples which penetrate the suture ring 195 and the exterior wall of the heart. The suture ring may be coupled to the universal heart port 100 (e.g., the first suture ring support flange 150 and/or the second suture ring support flange 160) in any suitable manner, such as with stitches, sutures, adhesives, mechanical fasteners, and/or the like. In embodiments in which the suture ring is coupled to the universal heart port with stiches, sutures, staple and/or the like, at least some of such fasteners may extend through one or more of the anchor cutouts 155, 165.

The anchor cutouts 165 may be configured to enable a surgeon (or other individual) to attach the universal heart port 100 to an outer surface of the heart or other organ through the use of sutures, surgical adhesives, surgical stapes, and/or the like. In this way, in using the first suture ring support flange 150 and the second suture ring support flange 160 to attach the universal heart port 100 to a suitable organ, at least a portion of the organ may be disposed within the organ receiving channel 170.

In the embodiment shown in FIG. 1, the universal heart port 100 further comprises a first adapter coupling flange 120A and a second adapter coupling flange 120B. Although in the embodiment shown in this figure, the universal heart port 100 comprises a first and second adapter coupling flange 120, it should be understood that in other embodiments, the universal heart port 100 may comprise any suitable number of adapter coupling flange 120. In various embodiments, each adapter coupling flange 120 comprises a substantially L-shaped protrusion that extends radially outward from the outer surface 114 of the body portion 105 adjacent the top surface 110. In this way, a portion of the top surface 110 extends into an upper portion of each adapter coupling flange 120, forming a substantially continuous top surface 110 that includes at least a portion of each adapter coupling flange 120. Each adapter coupling flange 120 comprises an end stop portion 125 that extends substantially perpendicularly downward from the substantially rectangular body of the adapter coupling flange 120. In various embodiments, the adapter coupling flange 120 extends from the end stop portion 125 to a leading edge 122. In various embodiments, each of the adapter coupling flange 120A and the adapter coupling flange 120B are disposed on opposing portions of the outer surface 114 of the body portion 105 and substantially evenly spaced about the outer surface 114 adjacent the top surface 110.

In particular embodiments, each adapter coupling flange 120 is configured to cooperate to maintain a suitable universal heart port adapter by engaging a corresponding geometry of the universal heart port adapter. In this way, the universal heart port 100 is configured to enable an individual to selectively couple and decouple a desired universal heart port adapter through use of each adapter coupling flange 120. This may, for example, enable a medical professional to utilize the universal heart port 100 in combination with any suitable desired equipment, to access a desired organ, or for any other suitable purpose.

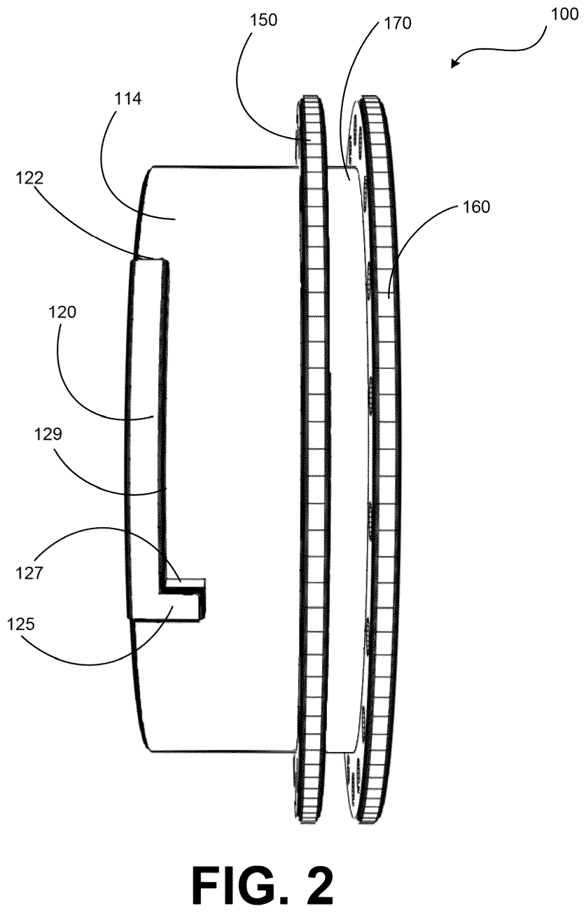

FIG. 2 depicts a side view of the universal heart port 100 and provides additional detail with respect to a particular embodiment of the adapter coupling flange 120. As may be understood from FIG. 2, the adapter coupling flange 120 further defines a flange support shelf engaging face 129 that is configured to engage and at least temporarily mate with a corresponding geometry of a suitable universal heart port adapter. The end stop portion 125 further defines an end stop inner face 127 that is substantially parallel to the leading edge 122.

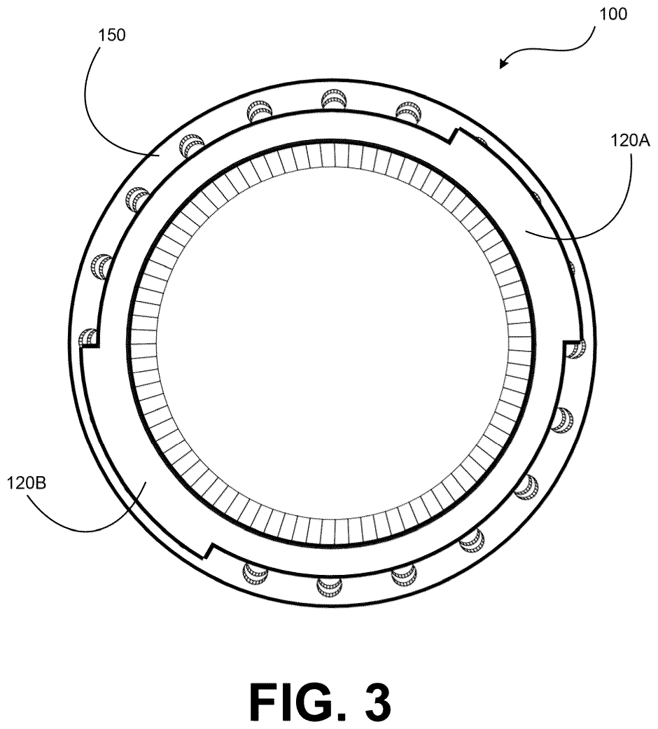

FIG. 3 depicts an overhead view of the universal heart port 100. As shown in FIG. 3, each adapter coupling flange 120 extends around a portion of the first suture ring support flange 150 along the 113. Although each adapter coupling flange 120 has been described as substantially L-shaped, it should be understood from FIG. 3 that each adapter coupling flange 120 is curved to correspond to an outer curve of the substantially cylindrical body portion 105.

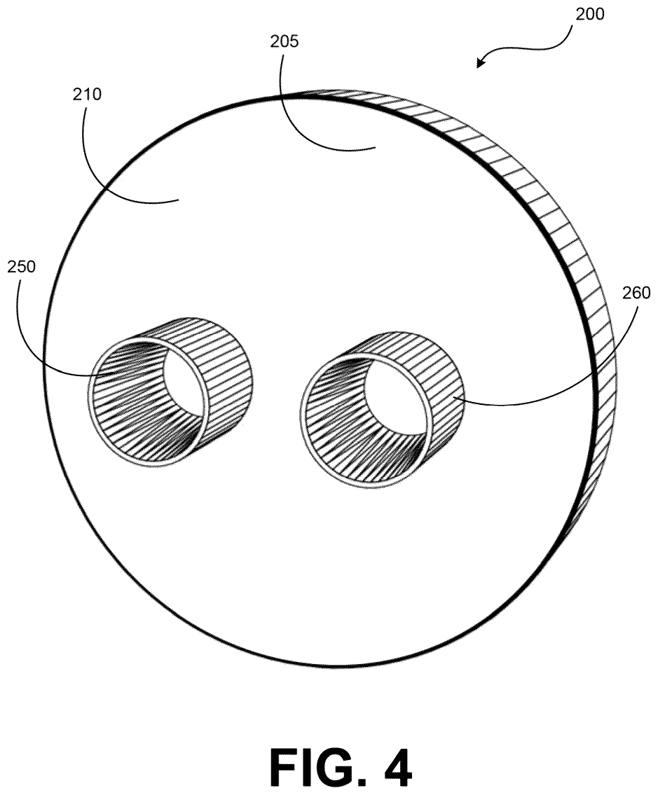

Turning to various embodiments of universal heart port adapters, FIG. 4 depicts a universal heart port adapter 200 according to a particular embodiment. In the embodiment shown in this figure, the universal heart port adapter 200 comprises a canula adapter. As may be understood from FIG. 4, the universal heart port adapter 200 comprises a substantially cylindrical body portion 205 defining a substantially circular top surface 210. Extending perpendicularly from the top surface 210 are a first locking membrane 250 and a second locking membrane 260. Each of the first locking membrane 250 and the second locking membrane 260 are substantially cylindrical and define an opening that extends centrally through the respective first locking membrane 250 and second locking membrane 260. In various embodiments, each of the first locking membrane 250 and the second locking membrane 260 are configured to engage a canula or other suitable tubing.

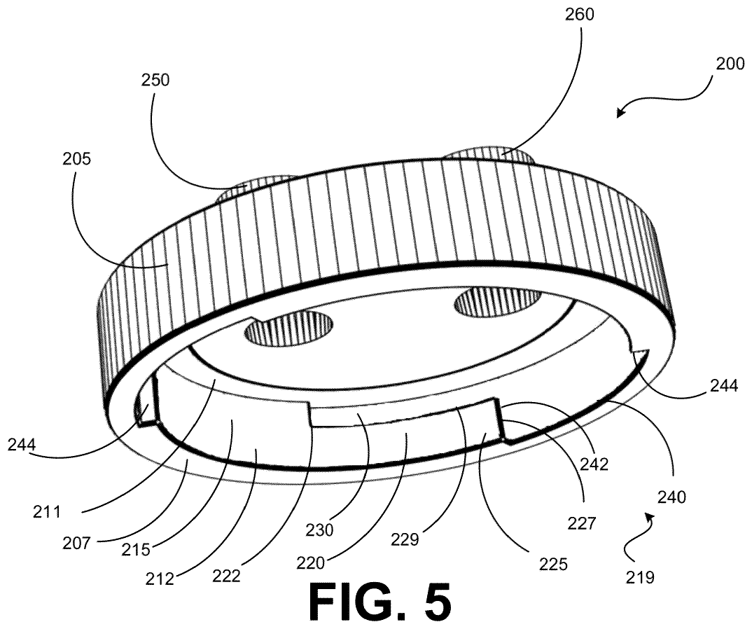

FIG. 5 depicts a bottom perspective view of the universal heart port adapter 200. As shown in this figure, the base portion of the universal heart port adapter 200 comprises a heart port engaging portion 219 that is configured to engage each adapter coupling flange 120 of the universal heart port 100 in order to at least temporarily couple the universal heart port adapter 200 to the universal heart port 100. It should be understood that the heart port engaging portion 219 described in the context of the universal heart port adapter 200 may be included adjacent the base of any suitable universal heart port adapter and/or plug, including those described herein. As will be understood from the additional detail provided below, the heart port engaging portion 219 is structured to correspond with a coordinating structure of the universal heart port 100 to provide a mechanism to selectively couple a desired universal heat port adapter to the universal heart port 100. As may be understood from FIG. 5, the body portion 205 defines a substantially circular cavity adjacent the base of the universal heart port adapter 200 upon which various components of the heart port engaging portion 219 are disposed.

As shown in FIG. 5, the structural aspects of the heart port engaging portion 219 are disposed in an interior portion of the cavity defined in the body portion 205. The heart port engaging portion 219 comprises a substantially rectangular flange support portion 215 that extends radially inward from an interior of the body portion 205 into the cavity defined in the body portion 205. The heart port engaging portion 219 further comprises a flange support shelf 220 that extends perpendicularly from the flange support portion 215 in a tangential direction about a portion of a perimeter of the interior cavity of the body portion 205 of the universal heart port adapter 200. As may be understood from this figure, the flange support portion 215 and the flange support shelf 220 form a substantially L-shaped support structure that is configured to engage at least a portion of the adapter coupling flange 120 shown in in FIG. 1 in order to maintain the universal heart port adapter 200 adjacent the universal heart port 100 when the universal heart port adapter 200 is coupled to the universal heart port 100.

The flange support portion 215 extends from a first end 242 adjacent the flange support shelf 220 to a second end 244. In the embodiment shown in FIG. 5, the universal heart port adapter 200 comprises a first flange support portion 215 and a second flange support portion 215 that are substantially evenly spaced about a circumference of an interior portion of the body portion 205. In various embodiments, the flange support portion 215 has a thickness that substantially corresponds to a thickness of the adapter coupling flange 120. The flange support shelf 220 defines a substantially rectangular end stop engaging portion 227 adjacent a shelf end portion 225 at the first end 242. In various embodiments, at least a portion of the end stop engaging portion 227 is configured to engage and/or mate with at least a portion of the end stop inner face 127 when the universal heart port 100 is coupled to the universal heart port adapter 200. In this way, the end portion of the flange support shelf 220 is configured to provide a rotational stopping mechanism to the universal heart port adapter 200 when the universal heart port adapter 200 is being coupled to the universal heart port 100 through interaction of the flange support shelf 220 and the shelf end portion 225.

In various embodiments, the body portion 205 defines a substantially circular heart port engaging face 211 that extends about a perimeter of an underside of the body portion 205 within the cavity defined by the body portion 205. As may be understood from FIG. 5, the flange support portion 215 extends from the heart port engaging face 211 to a bottom face 207 of the body portion 205. In some embodiments, the flange support portion 215 defines a flange leading edge engaging face 222 that extends perpendicularly from an upper portion of the flange support shelf 220 to the heart port engaging face 211. In some embodiments, the flange leading edge engaging face 222 has a height that substantially corresponds to a height of the leading edge 122 of the adapter coupling flange 120. In this way, the flange leading edge engaging face 222 may be configured to engage and at least temporarily mate with the leading edge 122 when the universal heart port adapter 200 is coupled to the universal heart port 100.

In various embodiments, the flange support shelf 220 defines a flange engaging face 229 adjacent an upper face of the flange support shelf 220. In various embodiments the flange engaging face 229 (e.g., and the flange support shelf 220) have a length that substantially corresponds to a length of the adapter coupling flange 120. In particular embodiments, the flange leading edge engaging face 222, the flange engaging face 229, and a least a portion of the heart port engaging face 211 define a flange receiving channel 230 configured to receive a corresponding adapter coupling flange 120 when the universal heart port adapter 200 is coupled to the universal heart port 100. In some aspects, the flange support portion 215 defines an inner face 212 disposed on the interior portion of the body portion 205. In some aspects, at least a portion of the inner face 212 is configured to engage a corresponding outer surface 114 of the universal heart port 100.

In various embodiments, the heart port engaging portion 219 defines a flange insertion channel 240 that extends from a first end 242 of a first flange support portion 215/flange support shelf 220 to a second end 244 of a second flange support portion 215/flange support shelf 220. In some aspects, the flange insertion channel 240 is configured to receive the adapter coupling flange 120 when the universal heart port adapter 200 is at least initially inserted into the universal heart port 100 for coupling. In various embodiments, the flange insertion channel 240 has a length that corresponds to a length of the adapter coupling flange 120.

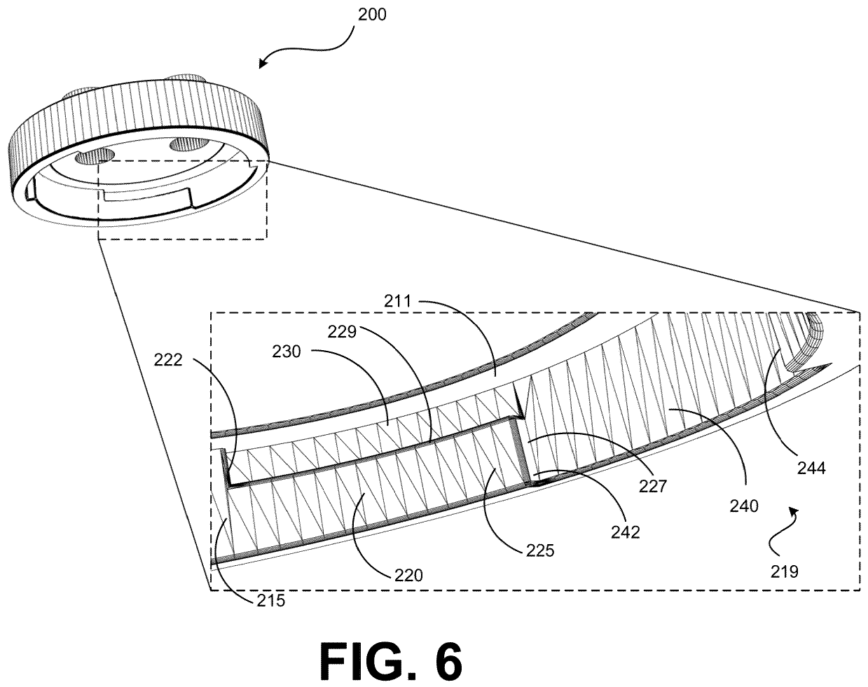

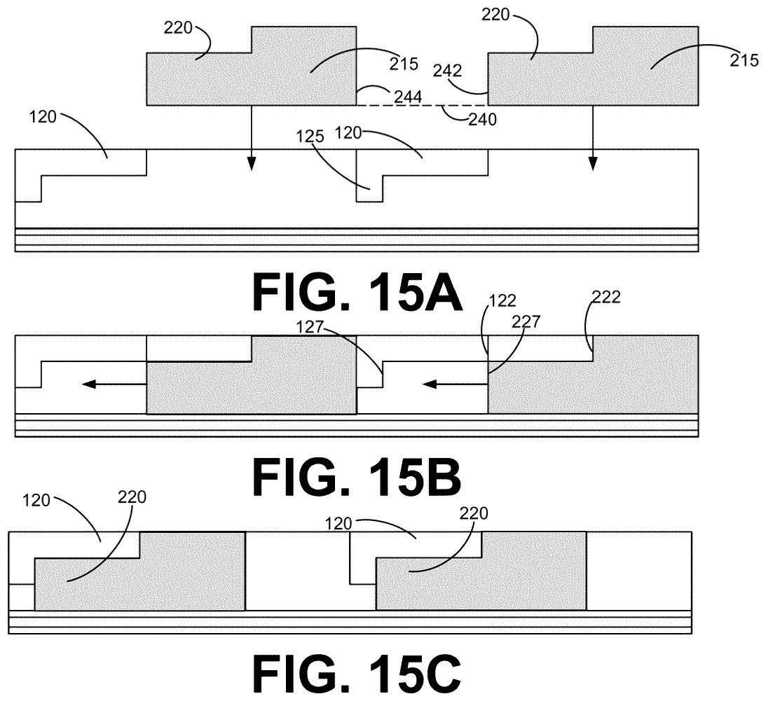

FIG. 6 is a detail view of the heart port engaging portion 219 on the underside of the universal heart port adapter 200. As may be further understood from FIG. 6, the heart port engaging portion 219 comprises at least one flange support shelf 220 that extends from an end of the flange support portion 215 adjacent the flange leading edge engaging face 222 to the shelf end portion 225 adjacent the first end 242 of the heart port engaging portion 219. Although the flange support shelf 220 has been described as substantially rectangular, it should be understood from these drawings that the flange support shelf 220 has a curve that corresponds to a circumference of the body portion 205. FIG. 6 depicts additional detail of the flange receiving channel 230 defined by the flange leading edge engaging face 222, the flange engaging face 229, and the heart port engaging face 211. As shown in FIG. 6, the flange receiving channel 230 is open to the flange insertion channel 240 adjacent the shelf end portion 225. In this way, the heart port engaging portion 219 is configured to enable a user to insert the adapter coupling flange 120 of a universal heart port 100 into the flange insertion channel 240 before rotating the adapter coupling flange 120 at least partially into the flange receiving channel 230 (e.g., as described more fully with respect to FIG. 15).

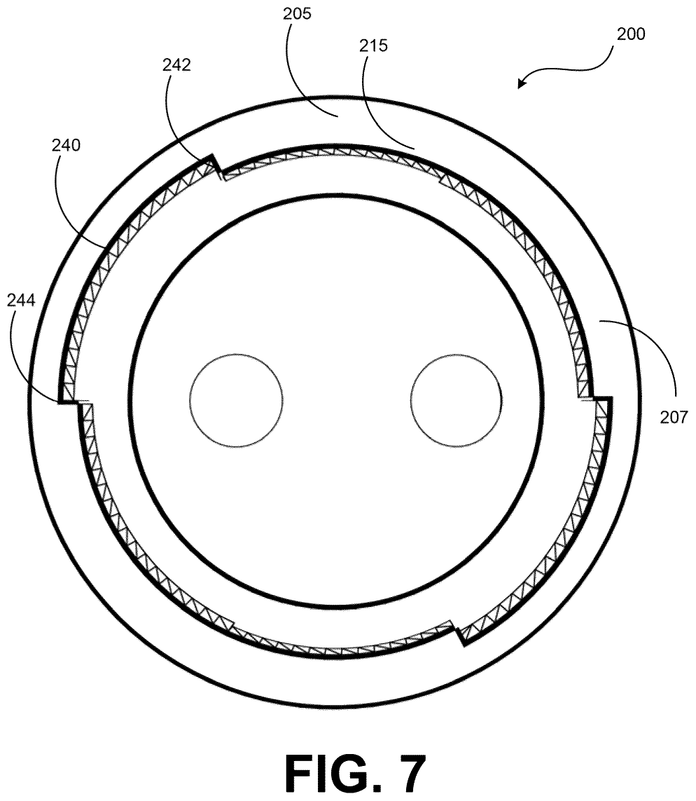

FIG. 7 depicts a bottom view of the universal heart port adapter 200 in accordance with various embodiments. As shown in this figure, each flange support portion 215 extends radially inward from the body portion 205 of the adapter about a portion of a circumference of a circular cavity defined in the base of the body portion 205. The flange support portion 215 (e.g., and the flange support shelf 220) is contiguous with the bottom face 207 of the body portion 205 such that the bottom face 207 and the flange support portion 215 form a substantially continuous bottom face of the universal heart port adapter 200. As further shown in this figure, various embodiments of the universal heart port adapter 200 comprise a first and second body portion 205 that are spaced apart by a respective first locking membrane 250 that extends from a first end 242 of a first flange support portion 215 to a second end 244 of a second flange support portion 215. In various embodiments, each flange insertion channel 240 is substantially the same size, shape, and orientation with respect to the universal heart port adapter 200.

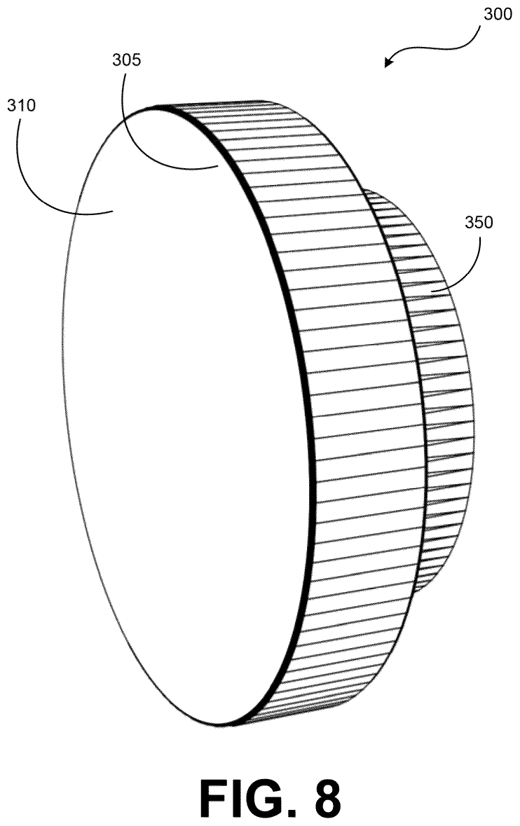

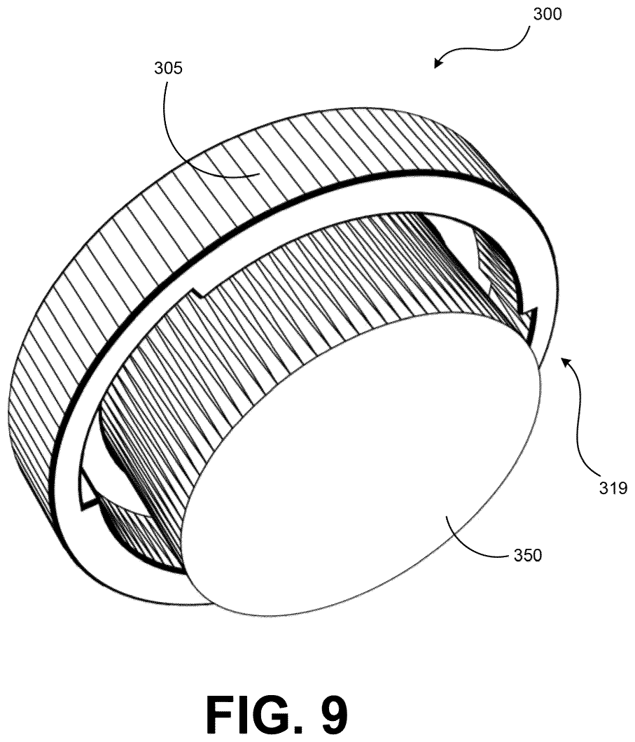

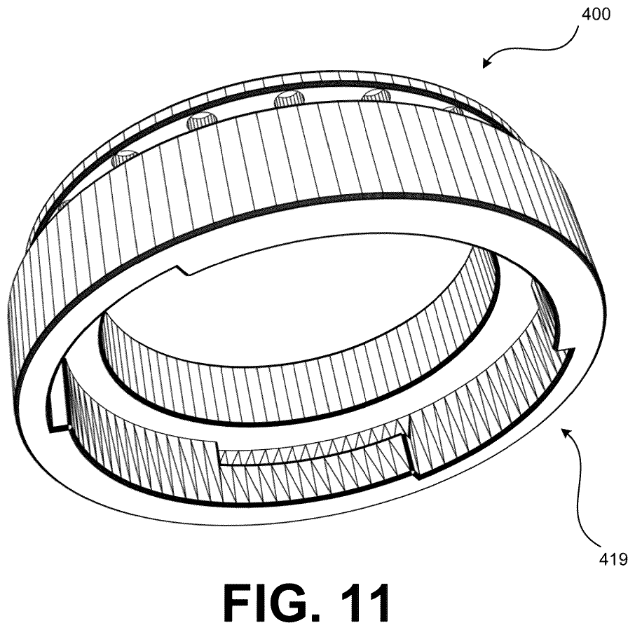

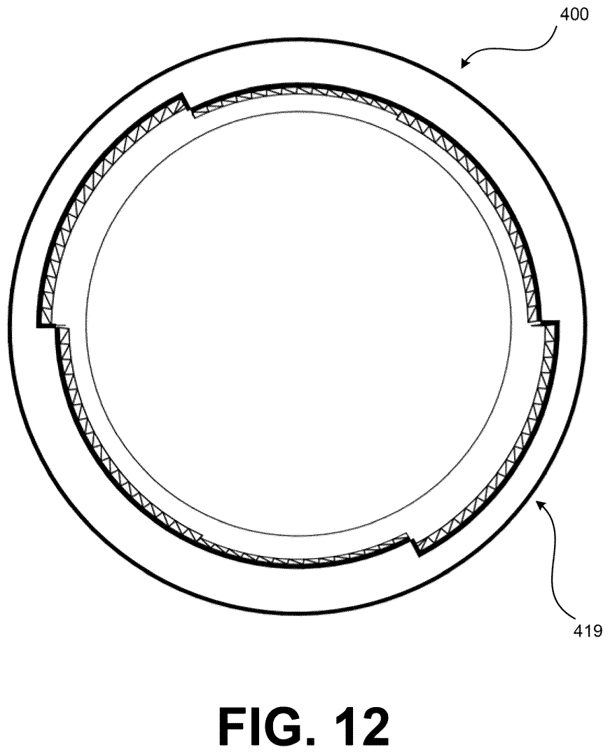

FIGS. 8-12 depict universal heart port adapters in accordance with various other embodiments. FIG. 8, for example, depicts a universal heart port plug 300 that includes a substantially cylindrical body portion 305 defining a substantially circular top surface 310. The universal heart port plug 300 further comprises a substantially cylindrical plug portion 350 that extends downward from the body portion 305 and has a radius that is smaller than a radius of the body portion 305. In various aspects, the radius of the plug portion 350 substantially corresponds to a radius of the membrane access cutout 111. In this way, when inserted into the universal heart port 100, the universal heart port plug 300 can plug an opening of the universal heart port 100 in order to substantially seal the heart port. FIG. 9 depicts a bottom view of the universal heart port plug 300. As may be understood from this figure, the universal heart port plug 300 may include a heart port engaging portion 319 is that is substantially structurally identical to the heart port engaging portion 219 described above (although not visible in FIG. 9 due to the plug portion 350). By utilizing structurally similar heart port engaging portions (e.g., heart port engaging portion 219, heart port engaging portion 319) in different embodiment of universal heart port adapters, the device may provide interchangeability of adapters utilizing the same heart port base (e.g., universal heart port 100).

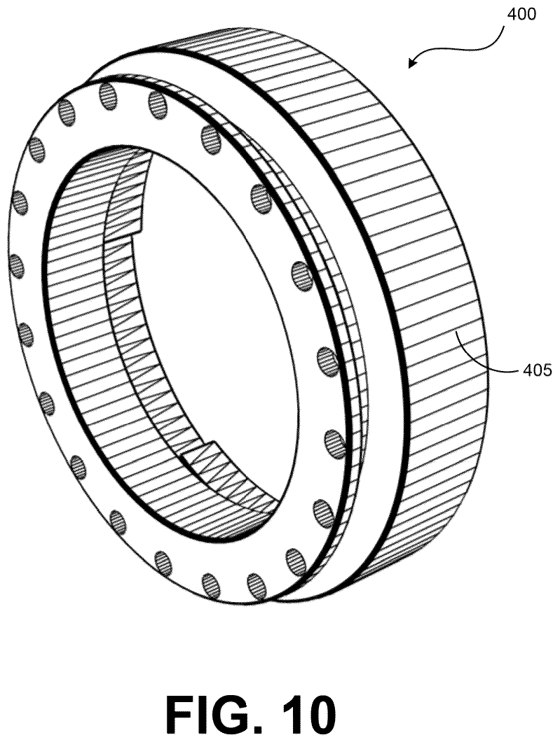

FIGS. 10-12 depict yet another embodiment of a universal heart port adapter. AS shown in this figure, a universal heart port adapter 400 may include a body portion 405 and be embodied as an LVAD adapter. In various embodiments, the body portion 405 may have any suitable length to accommodate various LVADs of different sizes, and for different patients. As with the universal heart port plug 300 discussed above, the universal heart port adapter 400 may have a hear port engaging portion 419 that is substantially structurally identical the universal heart port adapter 200 discussed herein with respect to the universal heart port adapter 200. By utilizing structurally similar heart port engaging portions (e.g., heart port engaging portion 219, hear port engaging portion 419) in different embodiment of universal heart port adapters, the device may provide interchangeability of adapters.

FIG. 13 depicts the universal heart port 100 attached to a heart 500. As may be understood from this figure, the universal heart port 100 may be affixed to the heart 500 in any suitable manner. For example, in various aspects, the universal heart port 100 may be sewn to the heart 500 using sutures 190 sewn through at least a portion of the suture ring 195). In various aspects, the universal heart port 100 may be sewn on the heart 500 prior to one or more incisions being made in the opening of the universal heart port 100 to access an interior of the heart 500. One or more adapters used in combination with the universal heart port 100 may then have access to an interior portion of the heart 500 (e.g., any suitable ventricle, chamber, etc.)

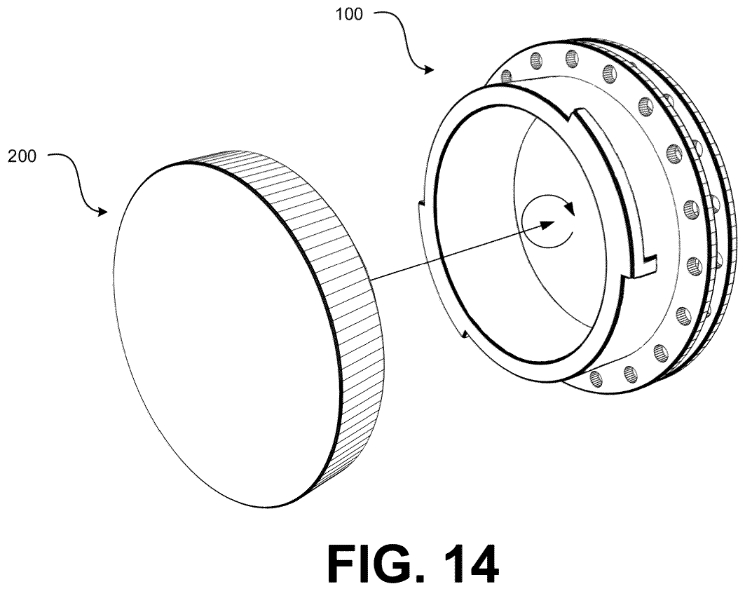

FIG. 14 depicts an exemplary process for coupling a universal heart port adapter 200 (e.g., or other heart port adapter) to a universal heart port 100. As may be understood from this figure, the universal heart port adapter 200 may be placed on the universal heart port 100 and rotated into place. FIGS. 15A-C depict a representative view of a side-cutaway view of a universal heart port adapter 200 being coupled to the universal heart port 100 and provide further detail of the interaction of the flange support shelf 220 and the adapter coupling flange 120 in order to maintain the universal heart port 100 adjacent the universal heart port adapter 200.

As may be understood from FIGS. 15A-C, when coupling the universal heart port adapter 200 to a universal heart port 100 (e.g., a universal heart port 100 that is affixed to a heart 500), a user may insert the universal heart port adapter 200 into the universal heart port 100 such that each of the first and second flange support portion 215 pass between each respective adapter coupling flange 120 of the universal heart port 100. As shown in FIG. 15A, each flange insertion channel 240 substantial corresponds in size to a length of the adapter coupling flange 120 (including the end stop portion 125). In this way, when the universal heart port adapter 200 is inserted into the universal heart port 100, the adapter coupling flange 120 passes between the first end 242 of a first flange support portion 215 and the first end 242 of a second flange support portion 215 through a flange insertion channel 240. The user continues inserting the universal heart port adapter 200 directly into the universal heart port 100 until any suitable portion of the universal heart port adapter 200 catches on any other suitable portion of the universal heart port 100. In this position, each leading edge 122 of each adapter coupling flange 120 is substantially co-facing and spaced apart from a flange leading edge engaging face 222 of a corresponding flange support portion 215. Similarly, each end stop inner face 127 of each adapter coupling flange 120 is substantially co-facing and spaced apart from at least a portion of a respective end stop engaging portion 227 of each flange support portion 215. As the user inserts the universal heart port adapter 200 into the universal heart port 100, as shown in FIG. 15B, each end stop engaging portion 227 clears a bottom of each corresponding leading edge 122. Once each end stop engaging portion 227 clears the bottom of each corresponding leading edge 122, the user can begin to rotate the universal heart port adapter 200 within the universal heart port 100 so that each adapter coupling flange 120 enters a corresponding flange receiving channel 230 and engages each flange support shelf 220. The user can continue to rotate the universal heart port adapter 200 until each end stop engaging portion 227 engages a corresponding end stop inner face 127 and until each flange leading edge engaging face 222 engages the corresponding leading edge 122 (e.g., as shown in FIG. 15C). In the position shown in FIG. 15C, the flange engaging face 229 of each flange support shelf 220 is engaging a corresponding adapter coupling flange 120, and each adapter coupling flange 120 is disposed between a corresponding flange engaging face 229, a corresponding flange leading edge engaging face 222, and the heart port engaging face 211 of the universal heart port adapter 200. In this position, each adapter coupling flange 120, through engagement with a respective flange support shelf 220, maintains the universal heart port adapter 200 in a mating relationship with the universal heart port 100 by preventing removal of the universal heart port adapter 200 without first reversing the rotation of the universal heart port adapter 200.

In particular embodiments, a user can reverse the steps shown in FIGS. 15A-15C in order to remove a particular universal heart port adapter, swap out for a second type of adapter, plug the universal heart port, etc. As such, once the universal heart port 100 has been affixed to a patient's heart 500 as shown in FIG. 13, the universal heart port 100 can provide a plurality of functionality and access to a surgeon through use of different adapters.

It should be emphasized that the above-described examples of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A universal heart port device comprising:
a universal heart port comprising:
a substantially cylindrical heart port body defining a substantially circular top surface, an outer surface, and a substantially cylindrical opening that extends through the heart port body;
a suture ring support flange that extends radially outward from the outer surface of the heart port body adjacent a base of the heart port body, the suture ring support flange being configured to support a suture ring adjacent the base of the universal heart port; and
one or more adapter coupling flanges, each respective adapter coupling flange of the one or more adapter coupling flanges extending radially outward along at least a portion of the top surface of the heart port body and comprising a respective end stop portion that extends perpendicularly downward from a respective end portion of the respective adapter coupling flange, wherein:
each adapter coupling flange is substantially parallel to and spaced apart from the suture ring; and
each of the one or more adapter coupling flanges are substantially evenly spaced about the outer surface of the heart port body.

2. The universal heart port device of claim 1, further comprising:
a universal heart port adapter comprising:
a substantially cylindrical body portion defining a substantially cylindrical cavity adjacent a base of the universal heart port adapter;
one or more heart port engaging portions disposed at least partially within the cavity, wherein each of the one or more heart port engaging portions comprise a respective flange support shelf configured to engage a respective adapter coupling flange of the one or more adapter coupling flanges and cooperate with the respective adapter coupling flange to at least temporarily couple the universal heart port to the universal heart port adapter.

3. The universal heart port device of claim 2, wherein the universal heart port adapter comprises at least one of a plug, a canula adapter, a left ventricular assist device (LVAD) adapter, an extension ring, or a membrane access port.

4. The universal heart port device of claim 2, wherein:
each of the one or more heart port engaging portions further comprise a respective flange support portion that extends from a base of the cavity to a heart port engaging face disposed adjacent an upper portion of the cavity in an interior of the substantially cylindrical body portion;
each respective adapter coupling flange extends perpendicularly from an end of a respective flange support portion adjacent the base of the cavity such that at least a portion of each respective adapter coupling flange, each respective flange support portion, and the heart port engaging face define a respective flange receiving channel configured to receive at least a portion of a respective adapter coupling flange.

5. The universal heart port device of claim 4, wherein each of the one or more heart port engaging portions are spaced apart by a distance that corresponds to a length of each respective adapter coupling flange including the respective end stop portion.

6. The universal heart port device of claim 4, wherein each respective flange receiving channel has a length that corresponds to a length of each respective adapter coupling flange without including the respective end stop portion.

7. The universal heart port device of claim 2, wherein:
the one or more adapter coupling flanges comprise a first adapter coupling flange and a second adapter coupling flange; and
the one or more heart port engaging portions comprise a first heart port engaging portion and a second heart port engaging portion, wherein:
the first adapter coupling flange is substantially structurally identical to the second adapter coupling flange; and
the first hear port engaging portion is substantially structurally identical to the second heart port engaging portion.

8. A universal heart port device comprising:
a base portion comprising:
a substantially cylindrical heart port body defining a substantially circular top surface, an outer surface, and a substantially cylindrical membrane cutout that extends through the heart port body;
a suture ring support flange that extends radially outward from the outer surface of the heart port body adjacent a base of the heart port body; and
one or more adapter coupling flanges, each respective adapter coupling flange of the one or more adapter coupling flanges extending radially outward along at least a portion of the top surface of the heart port body and comprising a respective end stop portion that extends perpendicularly downward from a respective end portion of the respective adapter coupling flange; and
an adapter comprising:
a substantially cylindrical body portion defining a substantially cylindrical cavity adjacent a base of the adapter; and
one or more base portion engaging portions disposed at least partially within the cavity, wherein each of the one or more base portion engaging portions comprise a respective flange support shelf configured to engage a respective adapter coupling flange of the one or more adapter coupling flanges and cooperate with the respective adapter coupling flange to at least temporarily couple the base portion to the adapter.

9. The universal heart port device of claim 8, wherein the adapter comprises at least one of a plug, a canula adapter, a left ventricular assist device (LVAD) adapter, an extension ring, or a membrane access port.

10. The universal heart port device of claim 8, wherein each of the one or more base portion engaging portions are spaced apart by a distance that corresponds to a length of each respective adapter coupling flange including the respective end stop portion.

11. The universal heart port device of claim 8, wherein:
each of the one or more base portion engaging portions further comprise a respective flange support portion that extends from a base of the cavity to a base portion engaging face disposed adjacent an upper portion of the cavity in an interior of the substantially cylindrical body portion; and
each respective adapter coupling flange extends perpendicularly from an end of a respective flange support portion adjacent the base of the cavity such that at least a portion of each respective adapter coupling flange, each respective flange support portion, and the base portion engaging face define a respective flange receiving channel configured to receive at least a portion of a respective adapter coupling flange.

12. The universal heart port device of claim 11, wherein each respective flange receiving channel has a length that corresponds to a length of each respective adapter coupling flange without including the respective end stop portion.

13. The universal heart port device of claim 8, wherein:
the one or more base portion engaging portions comprise a first base portion engaging portion and a second base portion engaging portion; and
the first base portion engaging portion and the second base portion engaging portion are substantially evenly spaced about an interior of the cavity.

14. The universal heart port device of claim 13, wherein the first base portion engaging portion and the second base portion define:
a first flange insertion channel between a first end of the first base portion engaging portion and a second end of the second base portion engaging portion; and
a second flange insertion channel between a second end of the first base portion engaging portion and a first end of the second base portion engaging portion.

15. The universal heart port device of claim 14, wherein each of the first flange insertion channel and the second flange insertion channel are dimensioned to receive a respective adapter coupling flange.

16. A method of affixing a universal heart port to a heart, the method comprising:
providing a universal heart port comprising:
a substantially cylindrical heart port body defining a substantially circular top surface, an outer surface, and a substantially cylindrical opening that extends through the heart port body;
a suture ring support flange that extends radially outward from the outer surface of the heart port body adjacent a base of the heart port body, the suture ring support flange supporting a suture ring that extends radially outward beyond an outer portion of the suture ring support flange; and
one or more adapter coupling flanges, each respective adapter coupling flange of the one or more adapter coupling flanges extending radially outward along at least a portion of the top surface of the heart port body and comprising a respective end stop portion that extends perpendicularly downward from a respective end portion of the respective adapter coupling flange;
using the suture ring to suture the universal heart port to the heart; and
creating an incision on the heart within at least a portion of the substantially cylindrical opening of the universal heart port.

17. The method of claim 16, further comprising:
providing a universal heart port adapter comprising:
a substantially cylindrical body portion defining a substantially cylindrical cavity adjacent a base of the universal heart port adapter;
one or more heart port engaging portions disposed at least partially within the cavity, wherein each of the one or more heart port engaging portions comprise a respective flange support shelf configured to engage a respective adapter coupling flange of the one or more adapter coupling flanges and cooperate with the respective adapter coupling flange to at least temporarily couple the universal heart port to the universal heart port adapter; and
at least temporarily coupling the universal heart port adapter to the universal heart port.

18. The method of claim 17, wherein at least temporarily coupling the universal heart port adapter to the universal heart port comprises:
inserting the universal heart port adapter into the universal heart port such that each of the one or more heart port engaging portions pass between adjacent adapter coupling flanges of the one or more adapter coupling flanges; and
rotating the universal heart port adapter until each respective end stop portion engages at least a portion of a respective end portion of each respective flange support shelf.

19. The method of claim 18, wherein rotating the universal heart port adapter until each respective end stop portion engages at least a portion of the respective end portion of each respective flange support shelf comprises rotating the universal heart port adapter such that each respective adapter coupling flange engages a respective flange support shelf.

20. The method of claim 16, wherein:
each of the one or more heart port engaging portions further comprise a respective flange support portion that extends from a base of the cavity to a heart port engaging face disposed adjacent an upper portion of the cavity in an interior of the substantially cylindrical body portion;
each respective adapter coupling flange extends perpendicularly from an end of a respective flange support portion adjacent the base of the cavity such that at least a portion of each respective adapter coupling flange, each respective flange support portion, and the heart port engaging face define a respective flange receiving channel configured to receive at least a portion of a respective adapter coupling flange; and
rotating the universal heart port adapter until each respective end stop portion engages at least a portion of the respective end portion of each respective flange support shelf comprises rotating the universal heart port adapter such that each respective adapter coupling flange is at least partially disposed within a respective flange receiving channel.
